(19) United States
(12) Patent Application Publication
(10) Pub. No.: US 2022/0211795 A1
(43) Pub. Date: Jul. 7, 2022

(54) CURCUMIN COMPOUND COMPOSITION AND METHOD FOR PREPARAING SAME

(71) Applicants: Chenland Nutritionals Inc., Irvine, CA (US); Qingdao Chenland Health Industry Group Co., Ltd., Qingdao (CN)

(72) Inventors: Shengcan Zou, Qingdao (CN); Shanglong Wang, Qingdao (CN); Lei Zong, Qingdao (CN); Jiancheng Zong, Qingdao (CN); Min Han, Qingdao (CN); Xin Li, Qingdao (CN); Mingliang Xu, Qingdao (CN)

(21) Appl. No.: 17/699,200

(22) Filed: Mar. 21, 2022

Related U.S. Application Data
(63) Continuation of application No. PCT/CN2021/141970, filed on Dec. 28, 2021.

(30) Foreign Application Priority Data
Oct. 21, 2021 (CN) 202111227566.4

(51) Int. Cl. A61K 36/9066; A61K 9/19; A61K 47/02; A61K 47/38

(52) U.S. Cl. CPC A61K 36/9066; A61K 47/38; A61K 47/02; A61K 9/19

(57) ABSTRACT

A curcumin compound composition and method for preparing the composition is disclosed. In order to improve the dissolution and bioavailability of curcumin, the compound composition is prepared by mixing curcumin and excipients according to a ratio, and the compound composition is prepared by tightly combining curcumin with glycerol monostearate and sodium carboxymethyl cellulose. The preparation process is suitable for industrialization, and the prepared compound composition has stable properties and improves the bioavailability of curcumin, and has good application and promotion prospects.

This patent application claims the benefit and priority of Chinese Patent Application No. 202111227566.4 filed on Oct. 21, 2021, the disclosure of which is incorporated by reference herein in its entirety as part of the present application.

### TECHNICAL FIELD

The present disclosure relates to a slow-release preparation of insoluble natural products, and more specifically, to a curcumin compound composition and method for preparing the composition.

### BACKGROUND ART

Curcumin (CUR), also known as curcumin or acid yellow, is a kind of polyphenol natural antioxidant extracted from the tubers of turmeric plants such as Curcuma longa, curcuma zedoary and curcuma turmeric, a diketone compound with the appearance of orange crystal powder, and it is a widely used food additive such as natural food pigments and condiments at home and abroad. At the same time, it has many pharmacological activities such as anti-inflammatory, anti-oxidation, anti-tumor, myocardial protection, anti-fibrosis, reducing blood lipid and anti-atherosclerosis, and it has been listed as the third generation anti-cancer chemopreventive agent by the National Cancer Institute of the United States.

However, Cur has low water solubility (11 ng/mL, 25° C.) and is unstable in nature (unstable in the presence of light, heat, strong acid bases, and some metal ions), in addition, it may be affect by CYP450, phase I metabolism enzyme in intestinal mucosa, Uridine diphosphate glucuronosyltransferase (UGTs) in intestinal tract, and drug efflux protein-P glycoprotein, which result in poor bioavailability in vivo after oral administration, which has seriously restricted its further development and promotion.

The main methods to solve the defects of curcumin are dosage form modification, homologous analogue modification and prodrug modification. In the basic research field of dosage form transformation, in view of the poor water solubility and low oral bioavailability of curcumin, the nano technology is mainly used to design pharmaceutical dosage forms, such as □nano-gel, □solid lipid nanoparticles, □ nano-emulsion, □nano-capsule, □nano-particle, □micelle, □microemulsion and □liposome.

In recent years, it is an effective method to study the parent structure of curcumin for prodrug design. The design of curcumin prodrug is mainly to esterify the phenolic hydroxyl group with small molecule or macromolecule carrier containing carboxyl group, by reforming phenolic hydroxyl group, on the one hand, the electronic transfer of enol structure is enhanced, the phenolic hydroxyl group is protected, the electronic delocalization in structure is eliminated, and the stability of curcumin derivatives is enhanced; on the other hand, the solubility of curcumin can be improved, the stability is improved, the half-life is prolonged, the bioavailability is improved, and the biological activity is enhanced by connecting small molecules or macromolecular carriers, at the same time, it has controlled release and sustained release effect, which is one of the effective strategies to solve the defects of curcumin application. At present, the research on curcumin prodrug abroad has made good progress and application, but the domestic research on curcumin prodrug is relatively few.

### SUMMARY

In view of this, the disclosure aims at solving the problems in the prior art and provides a curcumin compound composition and a method for preparing the composition.

In order to achieve the above purpose, the disclosure adopts the following technical scheme:

A curcumin compound composition, wherein the compound composition is composed of the following raw materials in terms of mass percent:

a curcumin 10˜60%;

a glyceryl monostearate 10˜60%;

a sodium carboxymethyl cellulose 10˜60%;

wherein, a viscosity of the sodium carboxymethyl cellulose is 50-2000 mPa·s.

It should be noted that the preparation means based on nanotechnology can improve the absorption of drugs, but the cost of nano-products is relatively high, and the stability and large-scale production are still difficult to transform, and are not suitable for further processing of raw materials. In addition, most of the existing reports focus on solid dispersion, inclusion technology and drug combination, although the absorption of drugs can be improved, and there is a certain prospect of industrialization. But from the overall point of view, the related research content is slightly vague and general, lack of clear and in-depth research corroboration. This is due to the fact that the absorption properties of curcumin itself are still unclear, in addition to its poor water solubility, instability and low bioavailability, there are no definite conclusions about the size of lipid solubility, the permeability of membrane, the main factors of instability, and the influence of efflux pump/metabolizing enzyme in and out of intestinal mucosa.

In addition, many studies only covered the preparation of pharmaceutical dosage forms in general and showed certain absorption improvement, but there are few studies on the reasons why dosage forms work and how to further optimize them, which also brought certain hidden troubles for future transformation work and practical application.

In view of this, we combined the previous research experience and modern preparation technology to conduct a detailed study and clear exposition on the improvement and optimization of pharmaceutical dosage forms.

Preferably, the compound composition is composed of the following raw materials in terms of mass percent:

a curcumin 30%;

a glyceryl monostearate 40%;

a sodium carboxymethyl cellulose 30%.

The disclosure also aims to provide a method for preparing the curcumin composite composition.

A method for preparing a curcumin compound composition, wherein the method is selected from a grinding and pulverizing method, a melting preparation method or a solvent drying method, and the above-mentioned preparation method aims to make the raw and auxiliary materials fully contact and uniformly disperse;

wherein,

the grinding and pulverizing method comprises the following steps: physically mixing the curcumin, the glycerol monostearate and the sodium carboxymethyl cellulose uniformly, grinding or pulverizing to form a solid compound;

the melting preparation method comprises the following steps: mixing the glyceryl monostearate and the sodium carboxymethyl cellulose, heating to a molten state, adding the curcumin to mixture for several times and stirring to obtain a combination spare; subsequently, further processing the combination spare to prepare the curcumin compound composition;

the solvent drying method comprises the following steps: mixing the curcumin and the glyceryl monostearate, adding an ethanol, and heating for dissolution to obtain A; dissolving the sodium carboxymethyl cellulose in room temperature water, and heating before use to obtain B; heating A and B, stirring, uniformly mixing, and rotary evaporating to remove the ethanol to obtain a curcumin dispersion; spray-drying or freeze-drying the curcumin dispersion to finally obtain a curcumin compound powder.

Preferably, a grinding and pulverizing equipment in the grinding and pulverizing method comprises, but is not limited to, a ball mill grinding equipment, a frozen pulverizing equipment; wherein, the frequency of the grinding equipment is 50-90 Hz, grinding is performed 1-5 times for 1-5 minutes, and an interval between two grinding is 10-20 s; a rotation speed of the ball mill equipment is 200-400 r/min, the time is 5-15 min, and the number of grinding is 1-5 times; in the frozen pulverizing equipment, the temperature of the freezen pulverizing is −20° C.˜−50° C., the feeding frequency is 8-20 Hz, the pulverizing frequency is 30-80 Hz, and the rotate speed of a fan is 30-80 Hz.

Preferably, a heating form of the combination spare is a water bath heating or a hot melting machine heating, wherein the water bath heating temperature is 60° C.-80° C., the stirring speed is 100-300 r/min, the stirring time is 60-80 min, the heating temperature is 80-120° C., the mixing time is 20-80 min, and the stirring speed is 80-300 r/min.

Further preferably, the further processing and preparation method of the obtained composition spare is a freeze pulverizing method, a spray drying granulation or an extrusion granulation.

Wherein, the freezing and pulverizing temperature is −20° C.˜−50° C., the feeding frequency is 8-20 Hz, the pulverizing frequency is 30-80 Hz, and the rotating speed of a fan is 30-80 Hz; the feeding speed of the spray drying granulator is 0.5 kg/h-2 kg/h, the temperature is −20° C.˜150° C., the feeding frequency is 8-20 Hz, the rotating speed of a fan is 30-80 Hz, and the rotating speed of an atomizing machine is 30-100 Hz; in the extrusion granulator, the material is continuously mixed at normal temperature for 5-10 min, heated at 42-62° C.; discharged at 3 rpm-100 rpm, the diameter of the extrusion hole is 0.50-3.00 mm, and the curcumin composite composition is obtained by pulverizing after extrusion.

Preferably, in the solvent drying method (A), the material ratio of the curcumin, glyceryl monostearate and ethanol is 10:1-20:1, and the heating temperature is 40° C.-70° C.; the material ratio of sodium carboxymethyl cellulose to water in (B) is (1-5):1, and the heating temperature before use is 50° C.-70° C.

Further preferably, the feeding speed of the liquid spray drying machine is 10-50 rpm, and the air inlet temperature is 35° C.˜100° C.

And, the freeze drying temperature is −80° C.-20° C., the vacuum pump pressure is 0.5-1 Pa, and the freezing time is 2-6 h.

As can be seen from the above technical scheme, compared with the prior art, the present disclosure provides a curcumin compound composition and method for preparing the composition, which have the following excellent effects:

A curcumin compound composition is disclosed. Glycerol monostearate, a food-grade adjuvant, is used as a lipid carrier to coat and disperse curcumin in a specific process (such as hot-melt extrusion, grinding, low-temperature pulverization or spray drying, etc.) to improve its solubility and dissolution in water, and suspending agents sodium carboxymethyl cellulose (CMC-Na) is added to improve the stability of the solution system. Compared with dosage forms such as liposomes and solid lipid nanoparticles adopted in the existing literature, the product has the advantages of simple prescription composition, suitable process for industrial production, stable product, small batch difference, low cost and convenience for promotion. Compared with other marketed products of curcumin, such as curcumin phospholipid compound and cyclodextrin inclusion compound, this product has higher drug loading (30%), good safety, low cost and high market promotion value. So far, there is no literature report or product launch that is similar to the design composition of this product.

### BRIEF DESCRIPTION OF THE DRAWINGS

In order to more clearly explain the embodiment of the disclosure or the technical scheme in the prior art, the following will briefly introduce the drawings needed in the description of the embodiment or the prior art, obviously, the drawings in the following description are only the embodiment of the disclosure, and for ordinary technicians in the field, other drawings can be obtained according to the provided drawings without paying creative efforts.

FIG. 1 is a particle size distribution diagram of curcumin compound 1.

FIG. 2 is a particle size distribution diagram of free curcumin.

FIG. 3 is a zeta potential distribution diagram of curcumin compound 1.

FIG. 4 is a zeta potential distribution diagram of free curcumin.

FIG. 5 is a standard graph of curcumin.

FIG. 6 is a comparison chart of solubility of curcumin compound 1 and free solution.

FIG. 7 is a release curve of curcumin compound 3 and free solution.

FIG. 8 is a fluorescence picture of curcumin cell uptake (a bright field picture of curcumin compound 3 groups; B curcumin compound 3 groups of dark field pictures; C control group bright field picture; D dark field picture of control group).

FIG. 9 is a diagram showing the toxicity test of curcumin and curcumin compound 1 on GL261 cells.

FIG. 10 is a scanning electron microscope test result of free curcumin (A magnified 500 times; B magnified 1500 times).

FIG. 11 is a scanning electron microscope test result of curcumin compound 1 (a magnified 1500 times; b magnified 5000 times).

FIG. 12 is a line chart showing the concentration-time changes of curcumin in Sabina C3 curcumin plasma.

FIG. 13 is a line chart showing the concentration-time changes of curcumin in curcumin compound 2 plasma.

FIG. 14 is a line chart showing the concentration-time changes of curcumin in curcumin compound 3 plasma.

FIG. 15 is a line chart showing the concentration-time changes of curcumin in curcumin compound 4 plasma.

FIG. 16 is a line chart showing the concentration-time changes of curcumin in curcumin compound 5 plasma.

FIG. 17 is a line chart showing the concentration-time changes of curcumin in curcumin compound 6 plasma.

### DETAILED DESCRIPTION OF THE EMBODIMENTS

The technical scheme disclosed by the disclosure will be clearly and completely described below in connection with the embodiments of the disclosure, and it is obvious that the described embodiments are only some embodiments of the disclosure, but not all embodiments. Based on the embodiments of the present disclosure, all other embodiments obtained by a person of ordinary skill in the art without creative work are within the scope of the present disclosure.

The embodiment of the disclosure discloses a curcumin compound with simple production process and stability and a method for preparing the compound, thereby greatly improving the bioavailability of curcumin.

For a better understanding of the present disclosure, the following examples are provided to further illustrate the disclosure, but are not to be construed as limiting the same, and non-essential modifications and adaptations of the disclosure by those skilled in the art in light of the foregoing summary are intended to be included within the scope of the present disclosure.

The technical scheme disclosed by the disclosure is further explained below with reference to specific embodiments.

### Embodiment 1: Preparation of Curcumin Compound Composition 1

Curcumin (95%, JIAHERB Phytochem), Soluplus (BASF), glyceryl monostearate and sodium carboxymethyl cellulose.

Weighing curcumin, glyceryl monostearate and sodium carboxymethyl cellulose, mixing them into 2 ml tubes matched with a grinding instrument at a mass ratio of 3:4:3, adding 2 small steel balls into each tube, shaking them well and putting them into a grinder at a frequency of 65 Hz, and milling twice for 1 minute each time with an interval of 10 s to obtain curcumin compound composition 1.

### Embodiment 2: Preparation of Curcumin Compound Composition 2

Curcumin (95%, JIAHERB Phytochem), Soluplus (BASF), glyceryl monostearate and sodium carboxymethyl cellulose.

Weighing 3 kg of curcumin, 4 kg of glyceryl monostearate, and 3 kg of CMC-Na, physically mixing well, and pulverizing by frozen pulverizer. The temperature of frozen pulverizing is −40° C., the frequency of feeding is 10 Hz, the pulverizing frequency is 50 Hz, and the speed of fan is 60 Hz. After room temperature recovery, the curcumin compound composition 2 is obtained by freeze drying and pulverization.

### Embodiment 3: Preparation of Curcumin Compound Composition 3

Curcumin (95%, JIAHERB Phytochem), Soluplus (BASF), glyceryl monostearate and sodium carboxymethyl cellulose.

Weighing curcumin, glyceryl monostearate and sodium carboxymethyl cellulose, mixing at a mass ratio of 3:4:3, heating at 80° C. in a water bath, stirring at a speed of 100 r/min, and stirring time for 60 min. The curcumin composite composition 3 is obtained by fully stirring and pulverization.

### Embodiment 4: Preparation of Curcumin Compound Composition 4

Curcumin (95%, JIAHERB Phytochem), Soluplus (BASF), glyceryl monostearate and sodium carboxymethyl cellulose.

Weighing curcumin, glyceryl monostearate and sodium carboxymethyl cellulose, mixing at a mass ratio of 3:4:3; then passing through a hot melt spray granulator, wherein the heating temperature of the hot melt spray granulator is 80° C.-120° C., the mixing time is 20 min-80 min, and the stirring speed is 80-300 r/min; the feeding speed is 20 rpm, the temperature is 80° C., the rotation speed of the blower is 50 Hz, and the rotation speed of the atomizer is 60 Hz to obtain the curcumin composite composition 4.

### Embodiment 5: Preparation of Curcumin Compound Composition 5

Curcumin (95%, JIAHERB Phytochem), Soluplus (BASF), glyceryl monostearate and sodium carboxymethyl cellulose.

Mixing 30 mg of curcumin with 40 mg of glyceryl monostearate, adding 1 ml of ethanol, and heating to dissolve at 50° C. to obtain A; dissolving 30 mg of sodium carboxymethyl cellulose in 30 ml of room temperature water, heating to 50° C. before use to obtain B; heating and uniformly stirring A and B, and evaporating rotary to remove ethanol to obtain curcumin dispersion; freeze-drying the curcumin dispersion to finally obtain curcumin composite composition 5.

### Embodiment 6: Preparation of Curcumin Compound Composition 6

Curcumin (95%, JIAHERB Phytochem), Soluplus (BASF), glyceryl monostearate and sodium carboxymethyl cellulose.

Mixing 3 kg of curcumin with 4 kg of glyceryl monostearate, adding 10 L of ethanol, and heating to dissolve at 50° C. to obtain A; dissolving 3 kg of sodium carboxymethyl cellulose in 3 L of room temperature water, heating to 50° C. before use to obtain B; Heating and uniformly stirring A and B, and evaporating rotary to remove ethanol to obtain curcumin dispersion; the dispersion-to-material ratio is 3:1, finally, the curcumin dispersion is passed through a low-temperature spray drying machine, the air inlet is 50° C., and the feeding speed is 20 rpm to obtain the curcumin composite composition 6.

The present disclosure is not limited to the contents of the above-described embodiments, but a combination of one or more of the embodiments may also achieve the object of the present disclosure.

In order to further verify the excellent effects of the present disclosure, the inventors also conducted the following experiments:

Experiment 1: Particle Size Potential

1.1 Test Samples

Curcumin compound composition 1 (curcumin 30 mg, glyceryl monostearate 40 mg, CMC-Na 30 mg)

Free curcumin: 0.0309 g curcumin+10 ml water to prepare a suspension

1.2 Particle Size Detection

Curcumin Compound Composition 1

Average particle size: 571d. nm PDI: 0.234 (FIG. 1).

Free Curcumin

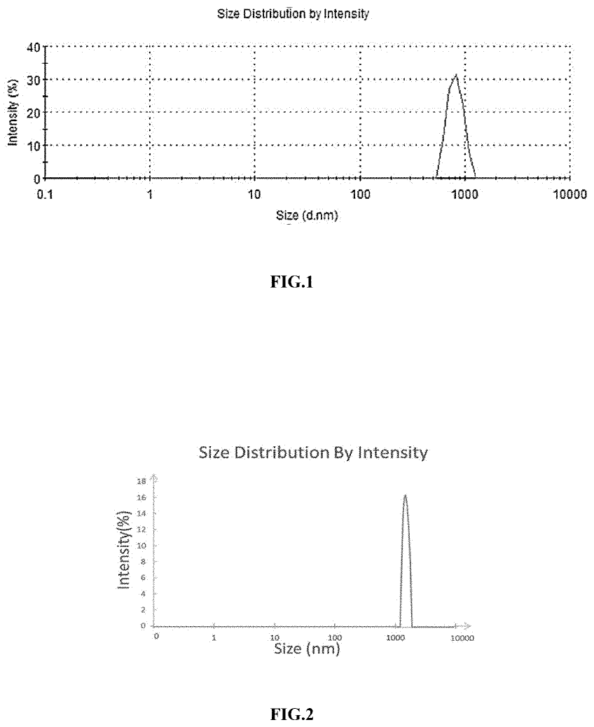

Hydrodynamic diameter: 4.053μm PDI: 0.545 (FIG. 2).

1.3 Potential Detection

Curcumin Compound Composition 1:

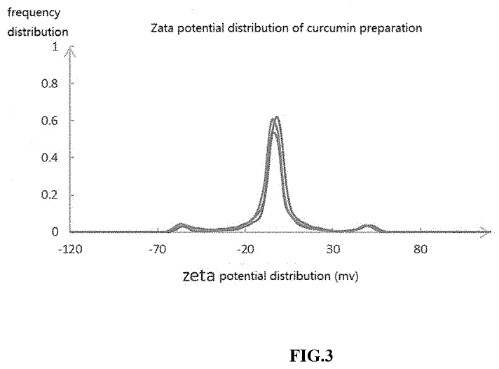

Average zeta potential: −3.5 mv (FIG. 3).

Free Curcumin:

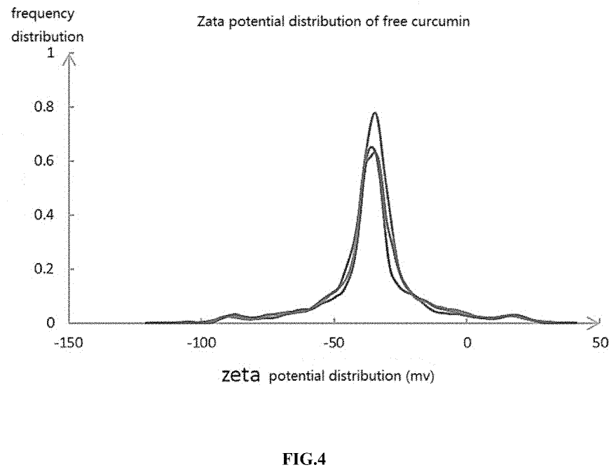

Average zeta potential: −36.5 mv (FIG. 4).

Experiment 2: Solubility Detection

2.1 Standard Curve Drawing

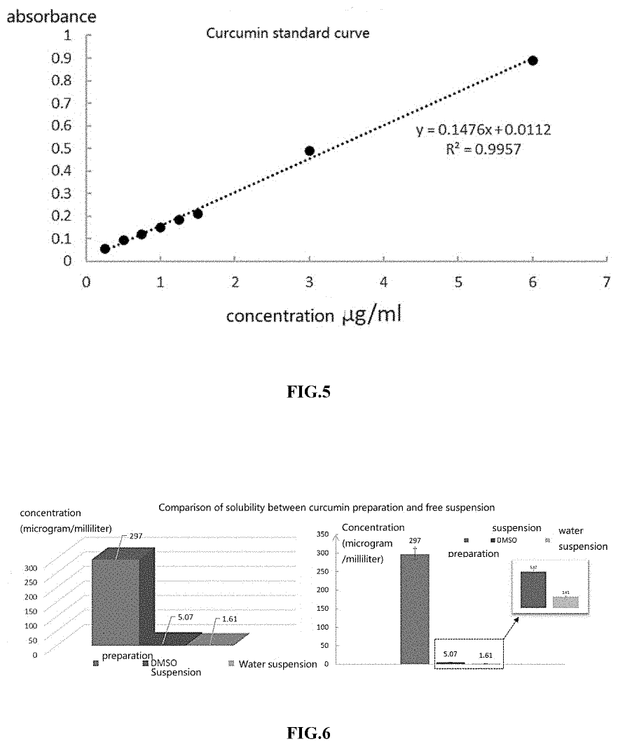

50 mg curcumin→50 ml volumetric flask, constant volume with an absolute ethyl alcohol, transfer 2.5 ml→50 ml volumetric flask, constant volume with an absolute ethyl alcohol to obtain a 0.05 mg/ml curcumin ethanol solution. Take 50, 100, 150, 200, 250, 300, 600 and 1200 μL of the above solutions respectively, add ethanol to make up to 10 ml to prepare 0.25, 0.5, 0.75, 1, 1.25, 1.5, 3 and 6μg/ml solutions with concentration gradients, and measure the absorbance at 425 nm, and draw a standard curve, as shown in FIG. 5.

TABLE 1
concentration absorbance corresponding
values of the standard curve

| | concentration μg/ml | | | | | | | |
|---|---|---|---|---|---|---|---|---|
| | 0.25 | 0.5 | 0.75 | 1.0 | 1.25 | 1.5 | 3.0 | 6.0 |
| absorbance | 0.056 | 0.094 | 0.122 | 0.15 | 0.183 | 0.21 | 0.491 | 0.887 |

According to the standard curve, the regression equation of the relationship between concentration and absorbance is y=0.1476x+0.0112, r=0.9978.

2.2 Solubility Detection

Sample:

1) shaking curcumin compound composition 1 well, and diluting a sample with water for 100 times for detection;

2) adding 30 mg of free curcumin with 10 ml of water to make a suspension, centrifuging at 4000 r for 10 min, and detecting the supernatant;

3) adding 50 mg of free curcumin and 40 ml of 1% DMSO into the suspension and centrifuging at 4000 r for 10 min for detection of the supernatant.

Test Results:

TABLE 2
Solubility test results

| | | Concentration | Actual concentration |
|---|---|---|---|
| sample | absorbance | μg/ml | μg/ml |
| compound 1 | 0.450 | 2.97 | 297 |
| water | 0.249 | 1.61 | 1.61 |
| suspension | | | |
| DMSO | 0.759 | 5.07 | 5.07 |
| suspension | | | |

As shown in FIG. 6, compared with two kinds of free suspensions, curcumin solubility in curcumin compound group 1 is greatly improved.

Experiment 3: Dissolution

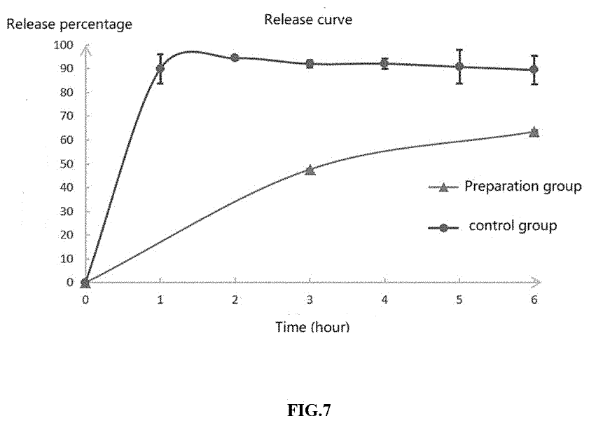

Detecting the drug release rate of the curcumin compound composition 4 at different time points, as shown in FIG. 7, compared with the free curcumin solution, the release rate of the curcumin compound composition 4 group shows a sustained release effect, and the cumulative release rate of the curcumin compound 4 group is about 60% in 6 h.

Experiment 4: Fluorescence Uptake by Curcumin Cells

4.1 Experimental Method

Culturing caco-2 cell in a six-well plate, and adhering caco-2 to the wall and growing under aggregation.

The concentration of free curcumin and curcumin compound composition 1 is 1.5 mg/ml, diluted with serum-free medium to 150 μg/ml, 1 ml per well, and incubated for 3 h in an incubator.

Remove the drug-containing culture solution, wash it with pbs three times, observe it under the microscope, and take photos. Fluorescence photography: Turn on the mercury lamp, adjust the intensity to the highest, select filter B, the curcumin spontaneous green fluorescence can be seen under the mirror, select automatic exposure in the software, take photos, add scale bar, and save.

4.2 Experimental Results:

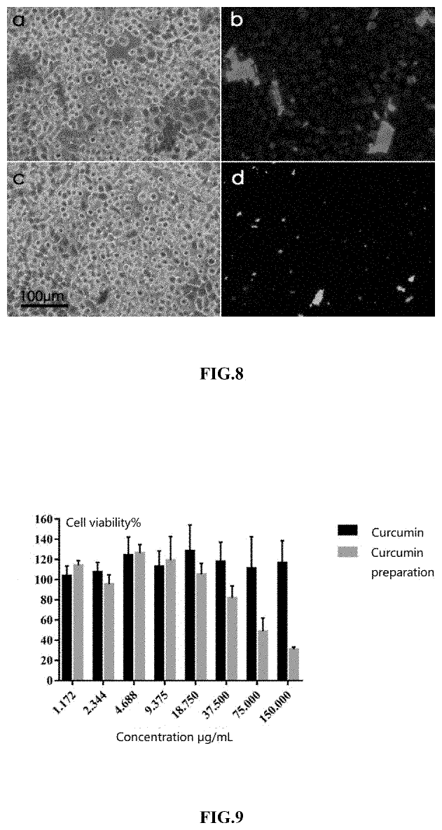

As can be seen from FIG. 8, the fluorescence uptake intensity of the cells in the curcumin compound 2 group (FIG. 8b) is significantly higher than that in the free curcumin group (FIG. 8d), indicating that the uptake of the cells in the curcumin compound 2 is significantly increased compared to the free curcumin group.

Experiment 5: Cytotoxicity Experiment

5.1 Experimental Method:

Gl261 cells in logarithmic growth phase are uniformly inoculated into sterile 96-well plates with 2×103 cells per well, and no cells are seeded around the plates, then 100 μL PBS is added into each well and cultured under the condition of 37° C. and 5% CO2 for 24 h; remove the culture medium and add 100 μL of drug-containing medium (concentration gradient set at 150.000 m/mL, 75.000 μg/mL, 37.500 μg/mL, 18.750 μg/mL, 9.375 μg/mL, 4.688 μg/mL, 2.344 μg/mL, and 1.172 μg/mL), the drug-containing medium is removed after culture for 4 h, and 100 μL of medium is added to each well for further culture for 20 h. The culture medium is removed, and culture medium containing 10% CCK8 is added. After incubation for 1 h, the absorbance is measured with a microplate reader, and the cell viability is determined by the following formula to cells.

$$Cell survival rate ( \% ) = \frac{{OD}_{Experimental} - {OD}_{blank}}{{OD}_{Contrast} - {OD}_{blank}} \times 100 \%$$

5.2 Experimental Results:

As shown in FIG. 9, the free curcumin has no obvious toxicity to glioma GL261 cells at the experimental concentration, while curcumin compound 1 has toxicity to GL21 cells at a certain concentration, especially at a high concentration, Showed better anti-tumor activity than free curcumin.

Experiment 6: Scanning Electron Microscope Examination

6.1 Experimental Method

The curcumin compound 6 and the free aqueous solution each 20 microliters are dropped onto a glass plate, air-dried naturally to prepare a detection sample, and after plating a conductive metal film, the sample is observed by a field emission sequence scanning electron microscope system, and photographed and recorded.

6.2 Experimental Results

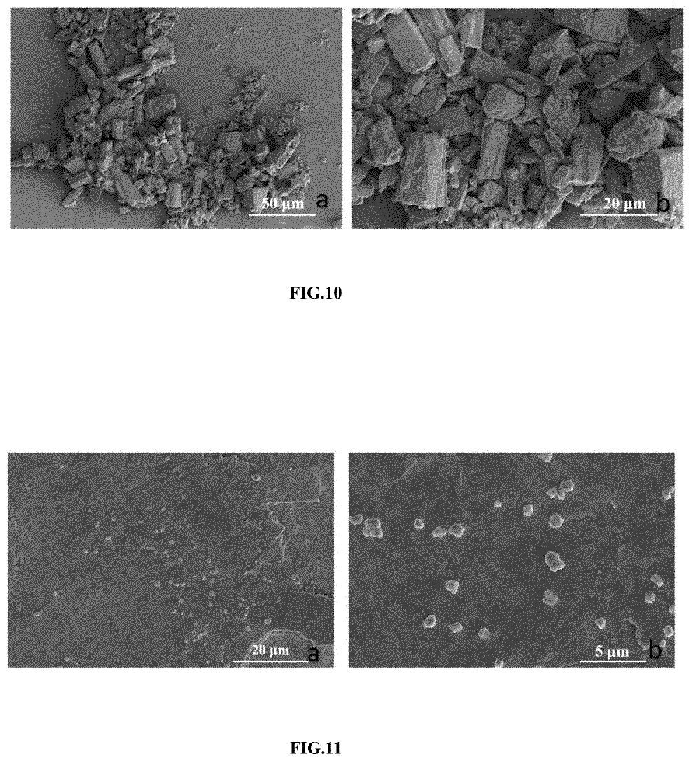

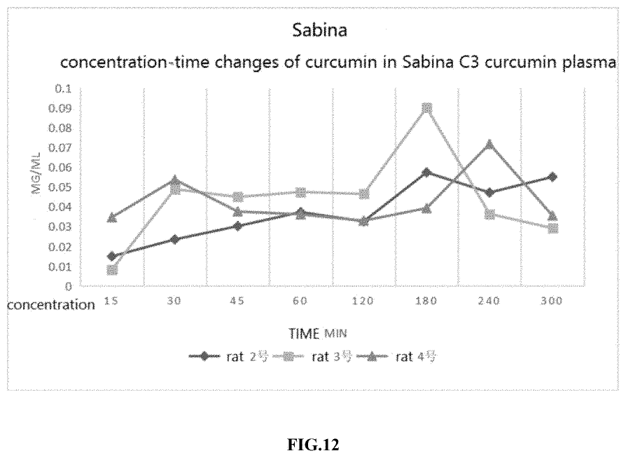

As can be seen from FIG. 10, the free curcumin is a micron-sized crystalline powder and is not uniform in size, and the curcumin compound 6 has more regular and uniform particle size distribution and particle shape, and the particle size distribution is mainly in the range of 100-1000 nm, which is significantly lower than the curcumin starting material, as shown in FIG. 11.

Experiment 7: Animal Experiments

7.1 Laboratory Animals

SD (Sprague-Dawley) rat, male, 200 g, purchased from Shanghai slack Experimental Animal Co., Ltd.

7.2 Dosing and Grouping

Grouping: Curcumin compound 2, Curcumin compound 3, Curcumin compound 4 prepared in Example, as well as the commercially available Sabinsha curcumin brand raw material C3, are given intragastrically with 4 rats in each group.

Dosing: The dosage is 200 mg/kg, 200 mg/kg, 200 mg/kg and 100 mg/kg respectively.

7.3 Blood Sample Processing

Sampling time of blood samples: 15 min, 30 min, 45 min, 1 h, 2 h, 3 h, 4 h, 5 h.

Method of blood collection and blood sample treatment: Orbital blood collection is performed on rats and blood samples were collected with heparinized 1.5 mL LEP tube (preparation method: dissolving proper amount of heparin sodium in ultrapure water to prepare 1% heparin sodium solution, putting 0.1 mL into 1.5 mL EP tube to soak the inner wall evenly, and putting into oven for drying), 0.5 mL of blood is collected from each tube. After centrifugation at 2000 rpm for 5 min, 150 μL of upper plasma was added with 100 μL nitrendipine ethyl acetate solution (250 μg/mL), followed by the addition of 1 ml ethyl acetate, which is vortexed and mixed for 2 min. After centrifugation at 12000 rpm for 5 min, the supernatant is aspirated. After the ethyl acetate is dried, it is re-dissolved with 100 μL methanol, vortexed for 30 s, and injected for determination by HPLC (high-pressure liquid chromatography) (injection volume of 20 μL). Wherein, the standard solution is treated in the same way.

7.4 Experimental Results

The calculated bioavailability of each compound relative to Sabine C3 curcumin is shown in the following table:

TABLE 3

| | | Dose | | |
|---|---|---|---|---|
| | AUClast | administered | Curcumin | Relative |
| Name | (min*μg/ml) | mg/kg | content % | bioavailability % |
| Sabina | 13.423 | 100 | 73.42 | |
| compound 2 | 20.916 | 200 | 22.43 | 255.03 |
| compound 3 | 16.9105 | 200 | 23.44 | 197.31 |
| compound 4 | 31.9238 | 200 | 23.07 | 378.45 |
| compound 5 | 11.932 | 200 | 20.46 | 159.50 |
| compound 6 | 18.5236 | 200 | 20.85 | 130.46 |

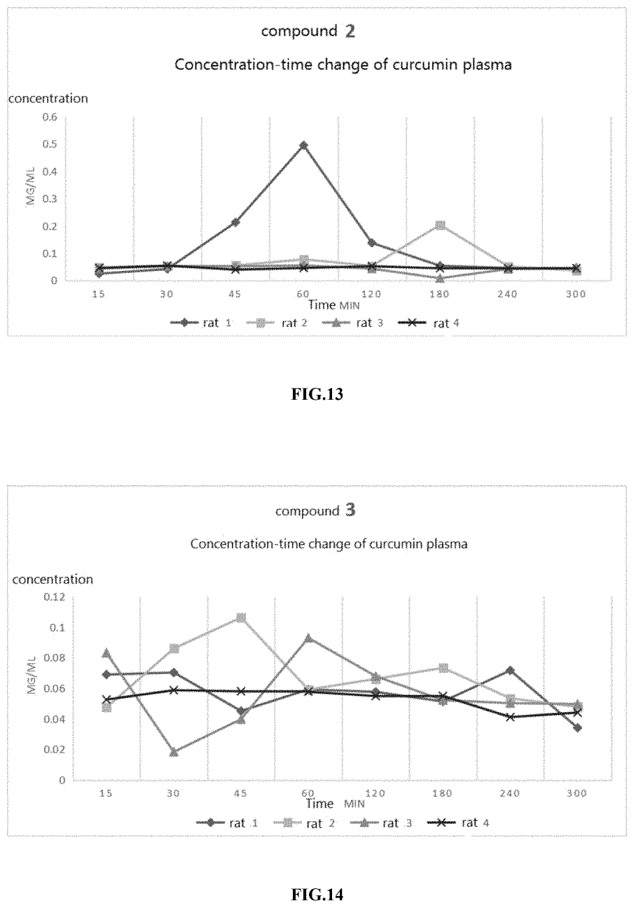

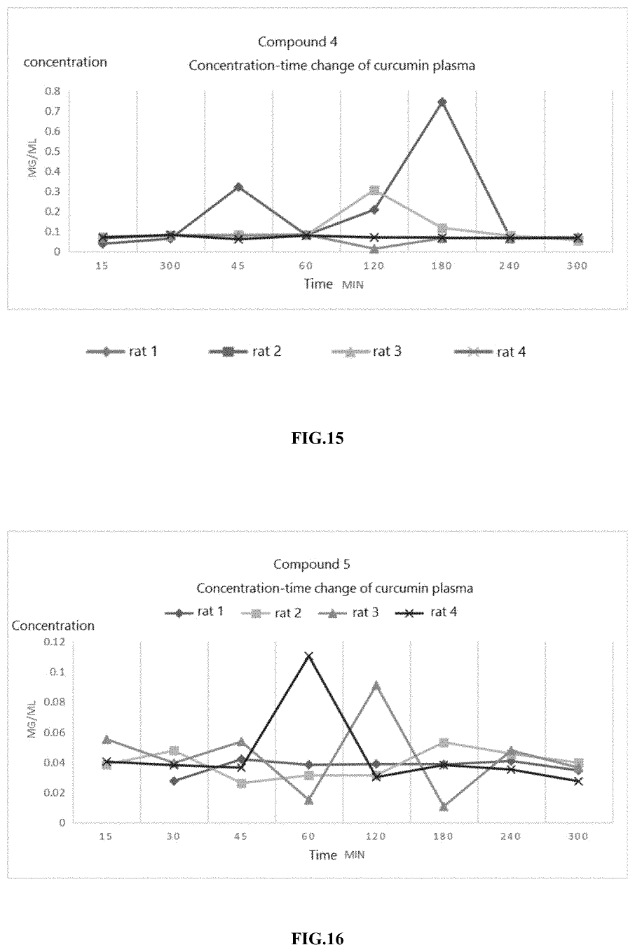

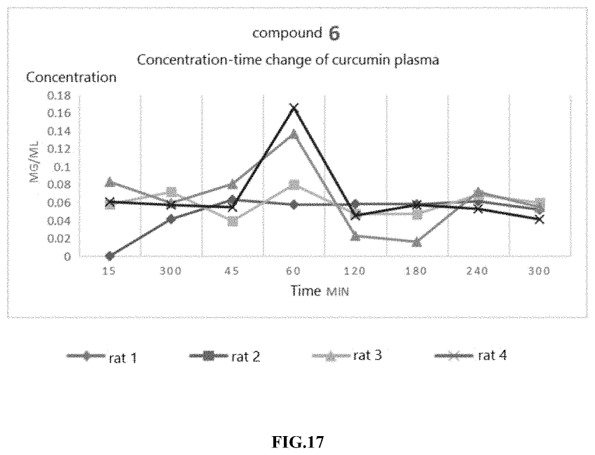

FIG. 12, FIG. 13, FIG. 14, and FIG. 15 are line charts of curcumin concentration-time changes in curcumin compound 2, curcumin compound 3, curcumin compound 4, and Sabina C3 curcumin plasma, respectively.

And compared with sabine C3, that relative bioavailability of the curcumin compound 2 groups is 255.03%, the relative bioavailability of the curcumin compound 3 group is 197.31%, the relative bioavailability of the curcumin compound 4 groups is 378.45%, the relative bioavailability of the curcumin compound 5 groups is 159.50%, and the relative bioavailability of the curcumin compound 6 groups is 130.46%, which proves that the bioavailability of the curcumin compound composition prepared by the disclosure is greatly improved.

The foregoing description of the disclosed embodiments enables those skilled in the art to make or use the present disclosure. Various modifications to these embodiments will be apparent to those skilled in the art, and the general principles defined herein may be used without departing from the spirit or scope of the disclosure, In other embodiment implementing that. Accordingly, the present disclosure will not be limited to the embodiments shown herein, but will be accorded the widest scope consistent with the principles and novel features disclosed herein.

What is claimed is:

1. A curcumin compound composition, wherein the compound composition is composed of the following raw materials in terms of mass percent:
a curcumin 10˜60%;
a glyceryl monostearate 10˜60%;
a sodium carboxymethyl cellulose 10˜60%;
wherein, a viscosity of the sodium carboxymethyl cellulose is 50-2000 mPa·s.

2. The curcumin compound composition of claim 1, wherein a composite composition is composed of the following raw materials in terms of mass percent:
a curcumin 30%;
a glyceryl monostearate 40%;
a sodium carboxymethyl cellulose 30%.

3. A method for preparing a curcumin compound composition, wherein the method is selected from a grinding and pulverizing method, a melting preparation method or a solvent drying method; wherein,
the grinding and pulverizing method comprises the following steps: physically mixing the curcumin, the glycerol monostearate and the sodium carboxymethyl cellulose uniformly, grinding or pulverizing to form a solid compound;
the melting preparation method comprises the following steps: mixing the glyceryl monostearate and the sodium carboxymethyl cellulose, heating to a molten state, adding the curcumin to mixture for several times and stirring to obtain a combination spare; subsequently, further processing the combination spare to prepare the curcumin compound composition;
the solvent drying method comprises the following steps: mixing the curcumin and the glyceryl monostearate, adding an ethanol, and heating for dissolution to obtain A; dissolving the sodium carboxymethyl cellulose in room temperature water, and heating before use to obtain B; heating A and B, stirring, uniformly mixing, and rotary evaporating to remove the ethanol to obtain a curcumin dispersion; spray-drying or freeze-drying the curcumin dispersion to finally obtain a curcumin compound powder.

4. The method of claim 3, wherein a grinding and pulverizing equipment in the grinding and pulverizing method comprises, but is not limited to, a ball mill grinding equipment, a frozen pulverizing equipment;
wherein, the frequency of the grinding equipment is 50-90 Hz, grinding is performed 1-5 times for 1-5 minutes, and an interval between two grinding is 10-20 s;
a rotation speed of the ball mill equipment is 200-400 r/min, time is 5-15 min, and the number of grinding is 1-5 times;
in the frozen pulverizing equipment, the temperature of the freezen pulverizing is −20° C.˜−50° C., the feeding frequency is 8-20 Hz, the pulverizing frequency is 30-80 Hz, and the rotate speed of a fan is 30-80 Hz.

5. The method of claim 3, wherein a heating form of the combination spare is a water bath heating or a hot melting machine heating, wherein the water bath heating temperature is 60° C.-80° C., the stirring speed is 100-300 r/min, the stirring time is 60-80 min, the heating temperature is 80-120° C., the mixing time is 20-80 min, and the stirring speed is 80-300 r/min.

6. The method for preparing the curcumin compound composition of claim 3, wherein the further processing and preparation method of the obtained composition spare is a freeze pulverizing method, a spray drying granulation or an extrusion granulation.

7. The method for preparing the curcumin compound composition of claim 5, wherein the further processing and preparation method of the obtained composition spare is a freeze pulverizing method, a spray drying granulation or an extrusion granulation.

8. The method of claim 6, wherein the freezing and pulverizing temperature is −20° C.˜−50° C., the feeding frequency is 8-20 Hz, the pulverizing frequency is 30-80 Hz, and the rotating speed of a fan is 30-80 Hz;
the feeding speed of the spray drying granulator is 0.5 kg/h-2 kg/h, the temperature is −20° C.˜150° C., the feeding frequency is 8-20 Hz, the rotating speed of a fan is 30-80 Hz, and the rotating speed of an atomizing machine is 30-100 Hz;
in the extrusion granulator, the material is continuously mixed at normal temperature for 5-10 min, heated at 42-62° C.; discharged at 3 rpm-100 rpm, the diameter of the extrusion hole is 0.50-3.00 mm, and the curcumin composite composition is obtained by pulverizing after extrusion.

9. The method of claim 3, wherein in the solvent drying method (A), the material ratio of the curcumin, glyceryl monostearate and ethanol is 10:1-20:1, and the heating temperature is 40° C.-70° C.; the material ratio of sodium carboxymethyl cellulose to water in (B) is (1-5): 1, and the heating temperature before use is 50° C.-70° C.

10. The method of claim 3, wherein the feeding speed of the liquid spray drying machine is 10-50 rpm, and the air inlet temperature is 35-100° C.

11. The method of claim 8, wherein the feeding speed of the liquid spray drying machine is 10-50 rpm, and the air inlet temperature is 35-100° C.

12. The method of claim 3, wherein the freeze drying temperature is −80° C.-20° C., the vacuum pump pressure is 0.5-1 Pa, and the freezing time is 2-6 h.
The method of claim 8, wherein the freeze drying temperature is −80° C.-20° C., the vacuum pump pressure is 0.5-1 Pa, and the freezing time is 2-6 h.
